(19) United States
(12) Patent Application Publication
(10) Pub. No.: US 2022/0226519 A1
(43) Pub. Date: Jul. 21, 2022

(54) PUSH BUTTON UNIT

(71) Applicant: OMRON CORPORATION, Kyoto-shi (JP)

(72) Inventors: Takuya KUDO, Kyoto-shi (JP); Takehiro AGATA, Kyoto-shi (JP)

(73) Assignee: OMRON CORPORATION, Kyoto-shi (JP)

(21) Appl. No.: 17/553,871

(22) Filed: Dec. 17, 2021

(30) Foreign Application Priority Data
Jan. 15, 2021 (JP) 2021-004738

(51) Int. Cl. A61L 2/10; G06F 3/02; A63F 13/24

(52) U.S. Cl. CPC A61L 2/10; G06F 3/0202; A61L 2202/14; A61L 2202/11; A63F 13/24

(57) ABSTRACT

A push button unit (10) comprises a push button (20), a shutter (31) that covers the push button (20) in a closed state, a shutter mechanism that opens and closes the shutter (31), and an ultraviolet light irradiation unit (40) that is disposed on the back surface of the shutter (31), is opposite an operation surface (20a) of the push button when the shutter (31) is closed, and has a light source (41) that emits a light beam that inactivates a virus.

### CROSS-REFERENCE TO RELATED APPLICATIONS

This application claims priority to Japanese Patent Application No. 2021-004738 filed on Jan. 15, 2021. The entire disclosure of Japanese Patent Application No. 2021-004738 is hereby incorporated herein by reference.

### BACKGROUND

### Technical Field

The present invention relates to a push button unit comprising a push button, and more precisely relates to a push button unit having a function of disinfecting the operation surface of the push button.

### Description of the Related Art

Some game machines installed in casinos, arcades, and other such game facilities are equipped with push buttons (for example, Patent Literature 1). Since these push buttons are operated by many different players, there is the risk that a virus that was clinging to the hand of a player who previously operated the push button will adhere to the operation surface of the push button. Therefore, in order for the next player to be able to play the game without worrying about any adhering virus, the employees of the game facility, etc., have to apply a disinfectant or the like to disinfect the operation surface frequently. In this specification, the word “disinfect” is used to mean “inactivate an adhering virus.”

The problem of viruses adhering to an operation surface is not limited to the push buttons of game machines, and the same applies to the push buttons provided to various other things such as elevators, vending machines, and ticket machines.

Also, push buttons have also been made of a material containing an antibacterial agent in the past (for example, Patent Literature 2).

### CITATION LIST

### Patent Literature

Patent Literature 1: JP-A 2016-214318
Patent Literature 2: JP-A H9-204560

### SUMMARY

However, frequent disinfection work increases the burden on employees, and disinfection using a disinfectant also has a problem in that it accelerates the deterioration of the push button. On the other hand, a push button made of a material containing an antibacterial agent does not require disinfection work by employees, and therefore does not impose any more work on the facility operator, nor is the push button deteriorated by a disinfectant. However, this approach is not as appealing to users as disinfection work performed by an employee, and the user may become anxious about the instantaneous efficacy of inactivating an adhering virus, or the disinfecting effect of a material containing an antibacterial agent.

It is an object of one mode of the present invention to provide a push button unit with which any virus adhering to an operation surface is inactivated without relying on manual disinfection work, which allows users to operate the push button with more peace of mind.

The present invention employs the following configuration in order to solve the above problem.

Specifically, the push button unit according to one aspect of the present invention comprises a push button, a shutter that covers the push button in its closed state, a shutter mechanism that opens and closes the shutter, and a light irradiation unit having a light source that is disposed on the back surface of the shutter, is opposite the operation surface of the push button in the closed state of the shutter, and emits a light beam that inactivates a virus.

With the above configuration, the light irradiation unit is disposed on the back surface of the shutter, which is opened and closed by the shutter mechanism. The light irradiation unit comprises a light source that emits a light beam that inactivates a virus, and is provided so as to be opposite the operation surface of the push button when the shutter is closed. Consequently, the drive of the shutter mechanism and the light irradiation unit can be controlled so that any virus adhering to the operation surface will be inactivated (killed) by the light beam emitted from the light irradiation unit, without relying on manual disinfection work. Also, since the light beam is emitted while the push button is covered by the shutter, the light beam can be prevented from leaking to the outside.

In the push button unit according to the above-mentioned aspect, the configuration may further comprise a panel having an opening through which the push button comes out, and an elevating mechanism that raises and lowers the push button, wherein the push button comes out through the opening at the raised position, and the shutter is disposed on the back surface of the panel and blocks off the push button into a closed state, with the push button in its lowered position.

With the above configuration, the elevating mechanism raises and lowers the push button, and the push button comes out through an opening formed in the panel at the raised position. The shutter is disposed on the back surface of the panel, and the opening is blocked off in a state in which the push button is in its lowered position, resulting in a closed state. Consequently, the shutter can have a flat configuration that slides on the back surface of the panel, and the push button unit can be thinner than with a configuration having a three-dimensional shape that covers the push button.

In the push button unit according to the above-mentioned aspect, the configuration may be such that the elevating mechanism comprises a motor and a pantograph mechanism that moves a base on which the push button is mounted between a lowered position and a raised position by means of the drive force of the motor. With the above configuration, the pantograph mechanism can be used to increase the reduction ratio of the gear that transmits the drive force of the motor to the pantograph mechanism, so that even if a strong force is exerted on the push button that has come out through the opening, the push button can be held in its raised position.

The push button unit according to the above-mentioned aspect have a configuration comprising an instruction receiving unit that receives, at an arbitrary timing, an instruction to execute a disinfection operation that inactivates any virus adhering to the operation surface with the light beam. With the above configuration, providing the instruction receiving unit makes it possible to disinfect the operation surface of the push button whenever desired.

In the push button unit according to the above-mentioned aspect, the configuration may be such that the instruction receiving unit comprises a reflection sensor. With the above configuration, since a reflection sensor is used, a user can instruct the execution of the disinfection of the operation surface in a non-contact manner.

The present invention is also a control device for the push button unit according to the above-mentioned aspect, wherein, when an event occurs that triggers a disinfection operation that inactivates any virus adhering to the operation surface occurs with a light beam, said control device controls the drive of the shutter mechanism to put the shutter in its closed state, controls the drive of the light irradiation unit when the shutter is in its closed state to turn on the light source for a specific length of time, and after this lighting is completed, controls the drive of the shutter mechanism to put the shutter in its open state.

With the above configuration, when an event occurs that triggers a disinfection operation, the shutter enters its closed state, the light source is turned on for a specific length of time in that state, and once this lighting of the light source is finished, the shutter enters its open state and the push button comes out. Consequently, setting an event that will trigger the execution of a disinfection operation allows the disinfection operation to be executed at the set point in time.

In the control device for the push button unit according to the above-mentioned aspect, when an event occurs that triggers the execution of the disinfection operation for inactivating a virus adhering to the operation surface with the light beam, the drive of the elevating mechanism is controlled to move the push button to its lowered position, and when the push button moves to its lowered position, the drive of the shutter mechanism is controlled to put the shutter in its closed state, and when the shutter enters its closed state, the drive of the light irradiation unit is controlled to turn on the light source for a specific length of time, and once the lighting of the light source is completed, the drive of the shutter mechanism is controlled to put the shutter in its open state, and when the shutter enters its open state, the drive of the elevating mechanism is controlled to move the push button to its raised position.

With the above configuration, when an event occurs that triggers a disinfection operation, the push button is lowered, the shutter enters its closed state, the light source is turned on for a specific length of time in that state, and after the lighting of the light source is completed, the shutter enters its open state and the push button rises and comes out. Consequently, setting an event that will trigger the execution of a disinfection operation allows the disinfection operation to be executed at the set timing.

### Effects

With one mode of the present invention, it is possible to realize a push button unit with which any virus adhering to the operation surface can be inactivated without relying on manual disinfection work, so the game can be enjoyed with more peace of mind.

### BRIEF DESCRIPTION OF THE DRAWINGS

FIG. 1 is an oblique view of a game machine in which a push button unit according to an embodiment is installed, showing a retracted state in which a push button has been retracted, and a normal state in which the push button has come out;

FIG. 2 is an oblique view of a push button unit installed in the game machine according to an embodiment, showing the normal state in which the push button has come out, and the retracted state in which the push button has been retracted;

FIG. 3 is an oblique view showing the inside of a front case in the push button unit installed in the game machine according to an embodiment, showing a state in which a shutter is open;

FIG. 4 is an oblique view showing the inside of the front case of the push button unit installed in the game machine according to an embodiment, showing a state in which the shutter is closed;

FIG. 5 is an oblique view showing the inside of the back case of the push button unit installed in the game machine according to an embodiment, showing a lowered state in which the push button is in its lowered position;

FIG. 6 is an oblique view showing the inside of the back case of the push button unit installed in the game machine according to an embodiment, showing a raised state in which the push button is in its raised position;

FIG. 7 is a detail view of the main components of the push button unit installed in the game machine according to an embodiment, at the portion where the left and right shutters abut against each other;

FIG. 8 is a schematic view showing the attachment of a timing belt and left and right sliders provided to a shutter mechanism in the push button unit installed in the game machine according to an embodiment;

FIG. 9 is an exploded oblique view of the push button in the push button unit installed in the game machine according to an embodiment;

FIG. 10 is a cross-sectional view of the main components, showing the lowered state and the raised state of a pantograph mechanism provided to an elevating mechanism in the push button unit installed in the game machine according to an embodiment;

FIG. 11 is an explanatory diagram showing the sensing of the position of the push button by third and fourth photosensors in the push button unit installed in the game machine according to an embodiment;

FIG. 12 is a block diagram showing a control system of the push button unit according to an embodiment;

FIG. 13 is a flowchart of the main sequence of the disinfection operation in the push button unit installed in the game machine according to an embodiment;

FIG. 14 is a flowchart showing the descent control sequence of the elevating mechanism in the push button unit installed in the game machine according to an embodiment;

FIG. 15 is a flowchart showing the control sequence of the shutter unit in the push button unit installed in the game machine according to an embodiment;

FIG. 16 is a diagram illustrating a push button unit according to a modification example, and is an oblique view showing a normal state in which the push button has come out, and a retracted state in which a push button has been retracted; and

FIG. 17 is a cross-sectional view of the main components of a push button unit comprising a plurality of push buttons according to a modification example, and shows a normal state in which the push buttons have come out and a retracted state in which the push buttons have been retracted.

### DETAILED DESCRIPTION

An embodiment relating to one aspect of the present disclosure (hereinafter, also referred to as “this embodiment”) will be described with reference to the drawings. In this embodiment, a configuration of a game machine such as a slot in which a push button unit is installed machine is given as an example of one mode of the push button unit disclosed herein.

### (1) Application Example

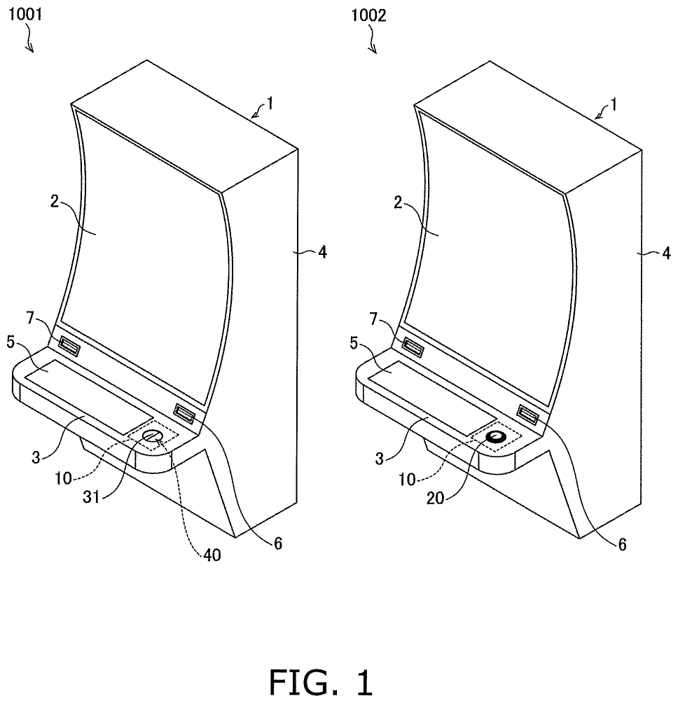

First, an example of a situation in which a push button unit 10 is applied will be described with reference to FIG. 1. FIG. 1 is an oblique view of the game machine 1 in which the push button unit 10 is installed in this embodiment, and shows a retracted state in which the push button 20 has been retracted, and a normal state in which the push button 20 has come out. 1001 in the drawing shows the retracted state, and 1002 shows the normal state.

As shown in FIG. 1, the game machine 1 is provided with a display monitor 2 at a position corresponding to the line of sight of the player at the upper part of the housing 4, and an operation unit 3 is provided below the display monitor 2. On the operation unit 3 are disposed an operation panel 5 constituted by an LCD and a touch panel, and a push button unit 10 comprising a push button 20. Also, an insertion slot 6 for inserting tickets or banknotes and a payout slot 7 for paying out tickets are provided between the display monitor 2 and the operation unit 3. Tickets and banknotes inserted into the slot 6 are sent to a ticket/banknote processing unit 8 (discussed below). Also, a ticket paid out from the ticket payout unit 9 is discharged from the payout slot 7.

In the game machine 1, the push button 20 is a button that is always operated in a game. For example, if the game executed by the game machine 1 is a slot machine game, the push button 20 is a spin button that begins the spin. Therefore, the push button 20 is touched much more frequently by the player than the operation panel 5, and the operation surface 20a of the push button 20 is a place where viruses and the like are most likely to adhere.

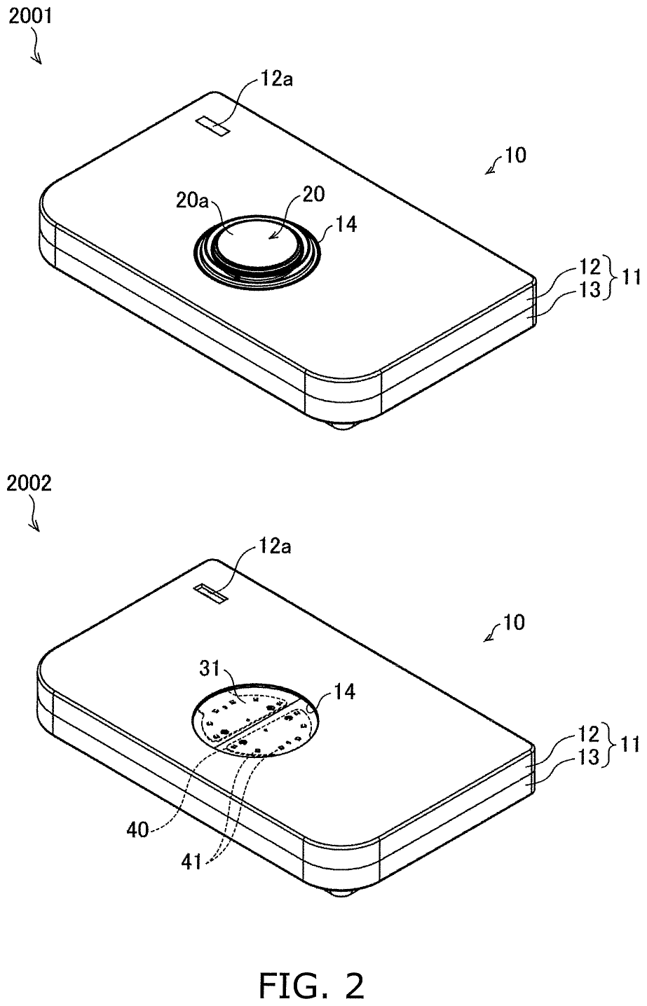

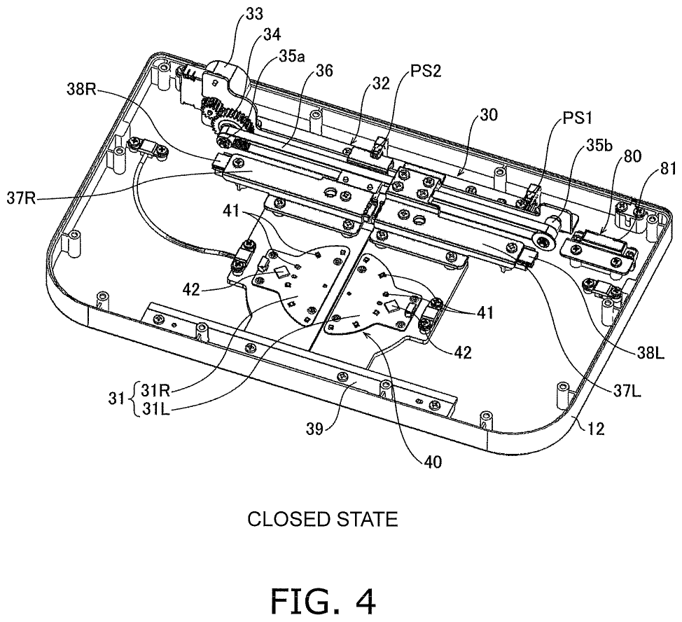

The push button unit 10 disclosed herein has a function of disinfecting the operation surface 20a of the push button 20. More specifically, as shown by 2002 in FIG. 2, the push button unit 10 comprising the push button 20 is designed such that the push button 20 retracts into a case 11, and an opening 14 is closed off by a shutter 31. As shown in FIG. 4, disinfecting LEDs 41 are disposed on the back surface of the shutter 31 and ultraviolet rays are emitted from the disinfecting LEDs 41 to inactivate any virus adhering to the operation surface 20a of the push button 20.

This frees employees from having to perform frequent disinfection work, and reduces the burden on the employees. Also, since no disinfectant solution, the operation surface 20a is not deteriorated by a disinfectant solution, and the service life of the push button 20 can be extended. Also, if the player is made aware that the operation surface 20a is disinfected in its retracted state, the player can confirm that the push button 20 has been disinfected whenever the retracted state is seen. Consequently, the notion of the button having been disinfected is more appealing to the player than a push button made of a material containing an antibacterial agent, and the player can rest assured that any virus has been inactivated.

### (2) Configuration Example

### 1. Overview of Push Button Unit 10

FIG. 2 is an oblique view of the push button unit 10 installed in the game machine 1 according to an embodiment, and shows a normal state in which the push button 20 has come out, and a retracted state in which the push button 20 has been retracted. 2001 in the drawing shows the normal state, and 2002 shows the retracted state.

As shown in FIG. 2, the push button unit 10 comprises a push button 20 and a case 11 for accommodating the push button 20. The case 11 is in the form of a box that is thinner in the pressing direction of the push button 20. The case 11 includes a front case 12 on which the operation surface of the push button 20 is located, and a back case 13 on which the push button 20 is installed. The push button unit 10 is installed in the operation unit 3 such that the upper surface of the front case 12 is flush with the a vanity panel surrounding the operation panel 5, for example (see FIG. 1). The upper surface of the front case 12 corresponds to a panel of the push button unit 10 in which is formed an opening 14 through which the push button 20 comes out.

In the case 11 is formed an opening 14 through which the push button 20 comes out. As shown by 2002 in the drawing, in the normal state the push button 20 has come out of the opening 14, and its operation surface 20a sticks out from the opening 14. On the other hand, as shown by 2002, in the retracted state the push button 20 has gone inside the case 11, and the opening 14 is closed off by the shutter 31.

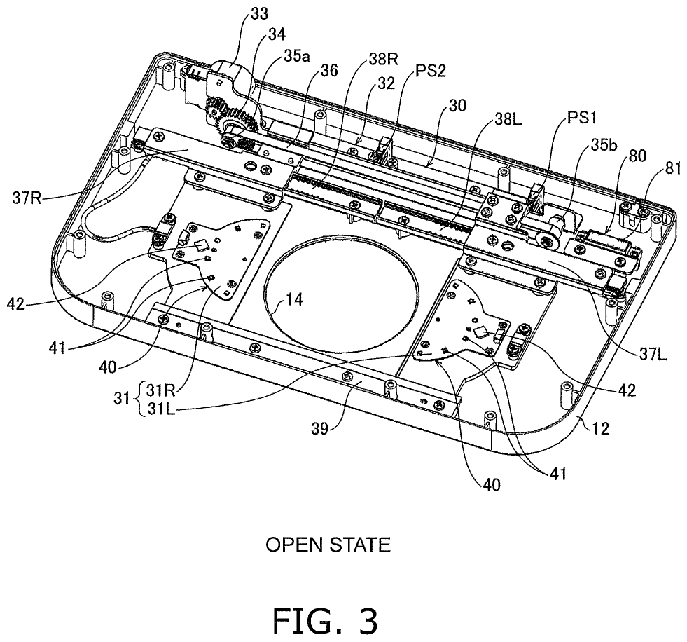

FIG. 3 is an oblique view showing the inside of the front case 12 of the push button unit 10 according to an embodiment, and shows a state in which the shutter 31 is open. FIG. 4 is an oblique view showing the inside of the front case 12 of the push button unit 10 according to an embodiment, and shows a state in which the shutter 31 is closed.

As shown in FIGS. 3 and 4, a shutter unit 30 and a sensor unit 80 are installed on the inside (back side) of the front case 12. The shutter unit 30 comprises a shutter 31 that closes off the opening 14, a shutter mechanism 32 that opens and closes the shutter 31, and an ultraviolet irradiation unit (light beam irradiation unit) 40 that is installed on the back surface of the shutter 31. As will be described in detail below, the ultraviolet irradiation unit 40 emits ultraviolet rays as a light beam for inactivating (killing) any virus adhering to the operation surface 20a of the push button 20. The sensor unit 80 receives an instruction from the outside to execute disinfection of the operation surface 20a with the ultraviolet irradiation unit 40.

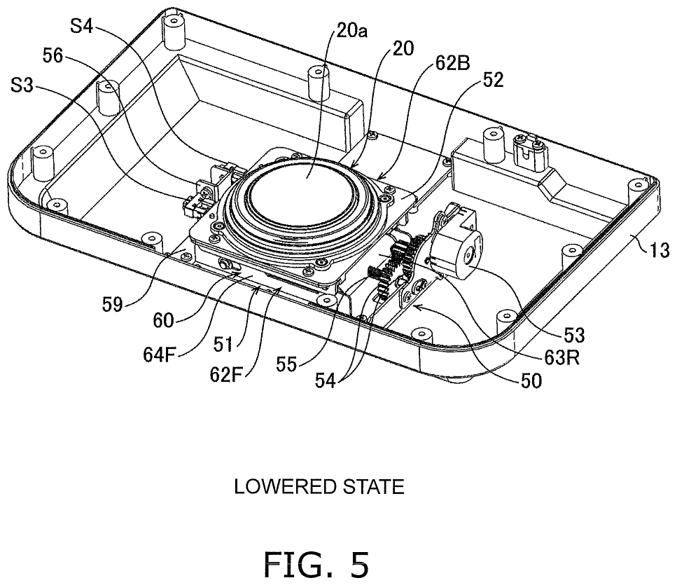

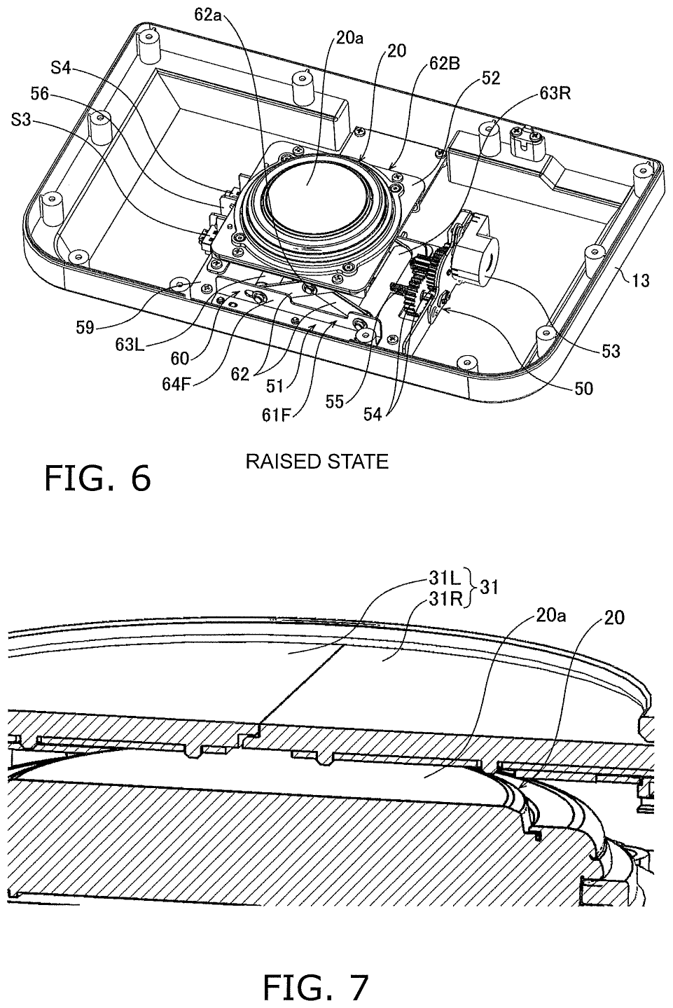

FIG. 5 is an oblique view showing the inside of the back case 13 of the push button unit 10 according to an embodiment, and shows a lowered state in which the push button 20 is in its lowered position. FIG. 6 is an oblique view showing the inside of the back case 13 of the push button unit 10 according to an embodiment, and shows a raised state in which the push button 20 is in its raised position.

As shown in FIGS. 5 and 6, an elevating unit 50 is installed on the inside (back side) of the back case 13. The elevating unit 50 is provided with the push button 20 and an elevating mechanism 51 for raising and lowering the push button 20.

### 2. Configuration of Shutter Unit 30

First, the configuration of the shutter unit 30 will be described. As mentioned above, the shutter unit 30 comprises the shutter 31, the shutter mechanism 32, and the ultraviolet irradiation unit 40. These members constituting the shutter mechanism 32 are unitized.

### 2-1. Shutter 31 and Shutter Mechanism 32

As shown in FIGS. 3 and 4, the shutter 31 is provided slidably on the back surface of the front case 12. In this embodiment, the shutter 31 consists of two shutters that move in opposite directions at the same time. For convenience of convenience in the following description, the push button unit 10 shall be assumed to be installed in the operation unit 3 such that the two-piece shutter 31 opens in the left-right direction and the sensor unit 80 is located to the rear. The shutter located on the left side when installed in the game machine 1 is referred to as the left shutter 31L, and the shutter located on the right side is referred to as the right shutter 31R. However, when it is not necessary to discuss the left and right shutters 31L and 31R separately, they shall be referred to simply as the shutter 31.

The left and right shutters 31L and 31R are opened and closed by the shutter mechanism 32, moving closer together during the transition from the open state to the closed state, and moving away from each other during the transition from the closed state to the open state. FIG. 7 is a detail view of the main components of the push button unit 10 according to an embodiment, at the portion where the left and right shutters 31L and 31R abut against each other. As shown in FIG. 7, the ends of the left and right shutters 31L and 31R that abut each other form a lap joint so as to overlap each other in plan view. This reliably prevents ultraviolet rays from the ultraviolet irradiation unit 40 (discussed below) from leaking out through the gap between the left and right shutters 31L and 31R in the closed state.

As shown in FIGS. 3 and 4, the shutter mechanism 32 comprises an opening and closing motor 33, a reduction gear 34, a pair of pulleys 35a and 35b, a timing belt 36, left and right sliders 37L and 37R, left and right rails 38L and 38R, a shutter rail 39, and first and second photosensors PS1 and PS2.

The opening and closing motor 33 is the drive source for opening and closing the shutter 31. The reduction gear 34 is a component that transmits the drive force of the opening and closing motor 33 to the timing belt 36. The drive shaft 33a of the opening and closing motor 33 engages with the reduction gear 34, and the reduction gear 34 is rotated by the drive force of the opening and closing motor 33. The pulleys 35a and 35b are members that rotate the timing belt 36, and the pulley 35a is fixed to the reduction gear 34. The timing belt 36 is wound around the pulleys 35a and 35b, and teeth formed on the pulley 35a on the drive side mesh with teeth formed on the inside of the timing belt 36.

The left and right sliders 37L and 37R and the left and right rails 38L and 38R are provided corresponding to the left and right shutters 31L and 31R. The left slider 37L is connected to the left shutter 31L and the timing belt 36. When the timing belt 36 rotates, the left slider 37L moves on the left rail 38L, causing the connected left shutter 31L to move. Similarly, the right slider 37R is connected to the right shutter 31R and the timing belt 36. When the timing belt 36 rotates, the right slider 37R moves on the right rail 38R, causing the connected right shutter 31R to move.

The ends of the left and right shutters 31L and 31R that are not connected to the left and right sliders 37L and 37R are supported slidably on the shutter rail 39.

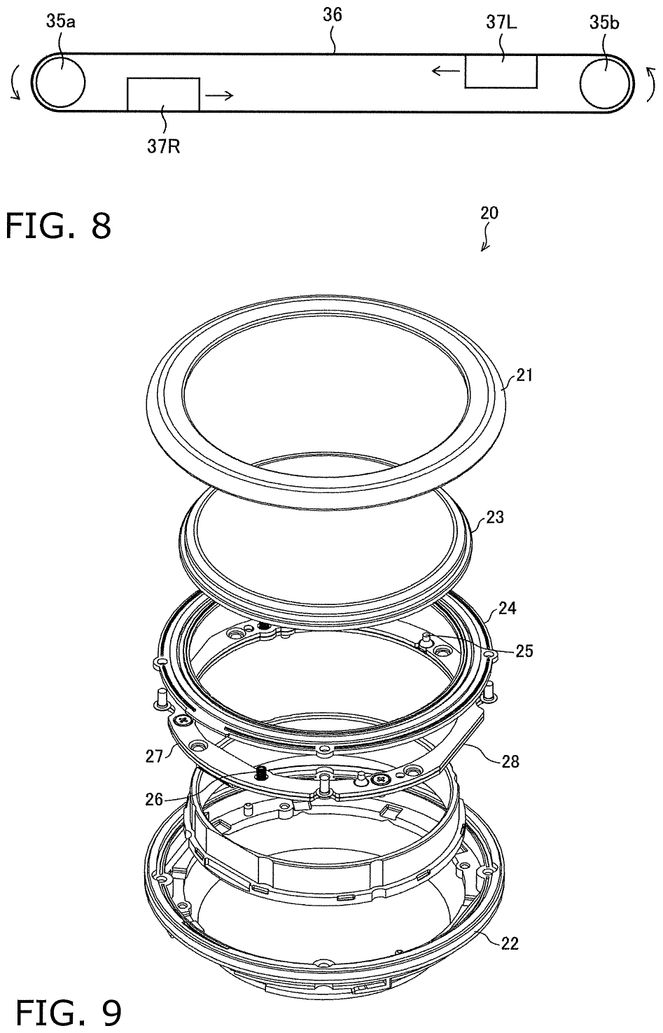

FIG. 8 is a schematic view showing the attachment of the timing belt 36 and the left and right sliders 37L and 37R included in the shutter mechanism 32 of the push button unit 10 according to an embodiment. As shown in FIG. 8, the left and right sliders 37L and 37R are attached in vertical symmetry at positions of half the circumference of the timing belt 36. Consequently, when the timing belt 36 rotates in the direction indicated by the arrows, the left and right sliders 37L and 37R move closer together, and the left and right shutters 31L and 31R move in the closing direction. Conversely, when the timing belt 36 rotates in the opposite direction from that indicated by the arrows, the left and right sliders 37L and 37R move away from each other, and the left and right shutters 31L and 31R move in the opening direction.

Returning to FIGS. 3 and 4, the first and second photosensors PS1 and PS2 detect whether the shutter 31 is open or closed. The first photosensor PS1 detects an open state (fully open state) in which the shutter 31 is open, and the second photosensor PS2 detects a closed state (fully closed) in which the shutter 31 is closed.

More specifically, the first photosensor PS1 is disposed at a position where light is not blocked until the left shutter 31L is fully opened (see FIG. 4), and where light is blocked by the left slider 37L when the left shutter 31L is fully opened (see FIG. 3). This allows the first photosensor PS1 to detect that the shutter 31 is in its open state when the light is being blocked.

On the other hand, the second photosensor PS2 is disposed at a position where light is not blocked until the right shutter 31R is completely closed (see FIG. 3), and where light is blocked by the right slider 37R when the right shutter 31R is fully closed (see FIG. 4). This allows the second photosensor PS2 to detect that the shutter 31 is in its closed state when the light is being blocked.

As described above, the left and right shutters 31L and 31R move symmetrically as the timing belt 36 rotates, so if the position of either of the left and right shutters 31L and 31R is detected, there is no need to detect the position of the other.

### 2-2. Ultraviolet Irradiation Unit 40

As shown in FIGS. 3 and 4, the ultraviolet irradiation unit 40 is installed on the back surface of the shutter 31 and emits ultraviolet rays as a light beam that inactivates any virus adhering to the operation surface 20a of the push button 20. The ultraviolet irradiation unit 40 comprises disinfecting LEDs (light sources) 41 that emit ultraviolet rays, and an LED drive circuit 42 that drives the disinfecting LED 41. The disinfecting LEDs 41 are disposed so that ultraviolet rays cover the entire operation surface 20a. The disinfecting LEDs 41 and the LED drive circuit 42 are installed on the substrate 43.

In this embodiment, the ultraviolet irradiation unit 40 is divided into two parts that are installed corresponding to the left and right shutters 31L and 31R. When the left and right shutters 31L and 31R are in their closed state, the disinfecting LEDs 41 face the operation surface 20a, and the ultraviolet rays emitted from the disinfecting LEDs 41 irradiate the operation surface 20a.

### 3. Configuration of Elevating Unit 50

Next, the configuration of the elevating unit 50 will be described. As discussed above, the elevating unit 50 comprises the push button 20 and the elevating mechanism 51 for raising and lowering the push button 20.

### 3-1. Push Button 20

FIG. 9 is an exploded oblique view of the push button 20 in the push button unit 10 according to an embodiment. As shown in FIG. 9, the push button 20 comprises a bezel 21, a base 22, a button body 23, a pressure-sensitive sensor substrate 27, an LED substrate 28, mechanical parts, and so forth. The bezel 21 is a cover member for covering (protecting) the outer peripheral part of the push button 20. The button body 23 is the portion that is pressed, and is supported between the bezel 21 and the base 22 so that the pressing operation can be performed. The upper surface of the button body 23 and the upper surface of the bezel 21 correspond to the operation surface 20a. The button body 23 is made of a transparent material, and an annular rubber portion 24 is fitted to the outer peripheral part thereof. The rubber portion 24 on the outer peripheral part is sandwiched between the bezel 21 and the base 22 so that liquid will not find its way inside the push button 20.

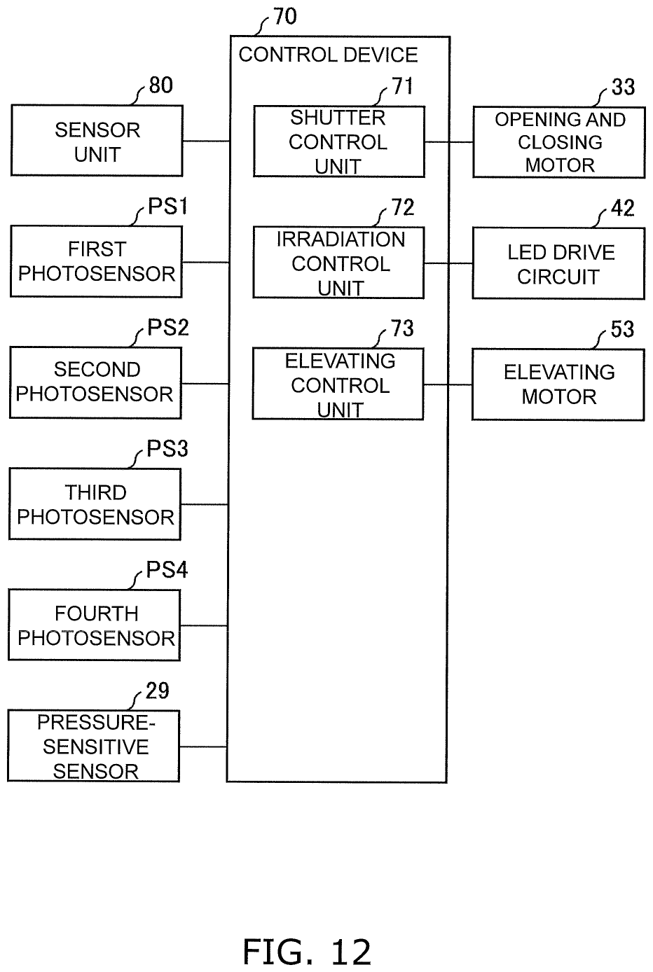

The mechanical parts include a plurality of rubber clickers 25 and a plurality of springs 26. The rubber clickers 25 give a tactile click when the button body 23 is pressed, and the spring 26 returns the pressed button body 23. The pressure-sensitive sensor substrate 27 comprises a pressure-sensitive sensor 29 (see FIG. 12) that detects the pressing of the button body 23, and therefore detects when the button body 23 is pressed. The LED substrate 28 comprises a plurality of LEDs (not shown) and illuminates the outer peripheral portion of the button body 23.

### 3-2. Elevating Mechanism 51

As shown in FIGS. 5 and 6, the push button 20 is installed on a base 52, and is raised and lowered along with the base 52 by the elevating mechanism 51. The elevating mechanism 51 comprises an elevating motor 53, two reduction gears 54, a ball screw 55, a pantograph mechanism 60, and third and fourth photosensors PS3 and PS4. The push button 20 and these members constituting the elevating mechanism 51 are assembled on the base plate to form a unit.

The elevating motor 53 is the drive source that raises and lowers the push button 20. The two reduction gears 54 are components that transmit the drive force of the elevating motor 53 to the ball screw 55. The two reduction gears 54 are rotated by the driving force of the elevating motor 53. One of the reduction gears 54 meshes with the drive shaft (not shown) of the elevating motor 53, and the ball screw 55 is fixed to the shaft of the other reduction gear. The reduction gears 54 are rotated by the drive force of the elevating motor 53. The ball screw 55 is a member that raises and lowers the pantograph mechanism 60.

The pantograph mechanism 60 has a plurality of frame pieces 62 arranged so as to intersect each other, and comprises a frame portion 61 on which an intersection point 62a is axially supported. In this embodiment, the frame portion 61 has two frame pieces 62, and also has a front frame portion 61F and a back frame portion 61B in the front-back direction intersecting the left-right direction, which is the opening and closing direction of the shutter 31.

The upper ends of each of the two frame pieces 62 in the front and back frame portions 61F and 61B are attached to the base 52, and the base 52 is supported by the four frame pieces 62. The lower ends of the two frame pieces 62 in the front and back frame portions 61F and 61B are attached to the shafts of round support columns 63R and 63L disposed on the left and right. More specifically, the lower ends of the two frame pieces 62 of the front frame portion 61F are attached to the front shafts of the left and right round support columns 63L and 63R, and the lower ends of the two frame pieces 62 of the back frame portion 61B are attached to the rear shafts of the left and right round support columns 63L and 63R.

The shafts at both ends of the left and right round support columns 63L and 63R are supported by a front support plate 64F and a back support plate 64B installed in the front-back direction. Of these, the shaft of the right round support column 63R is received at a fixed position by the front and back support plates 64F and 64B. The shaft of the left round support column 63L is slidably received by slots 64a that are formed in the front and back support plates 64F and 64B and are longer in the left-right direction. The ball screw 55 is disposed so as to pass through the center of the left and right round support columns 63R and 63L in the lengthwise direction (front-back direction).

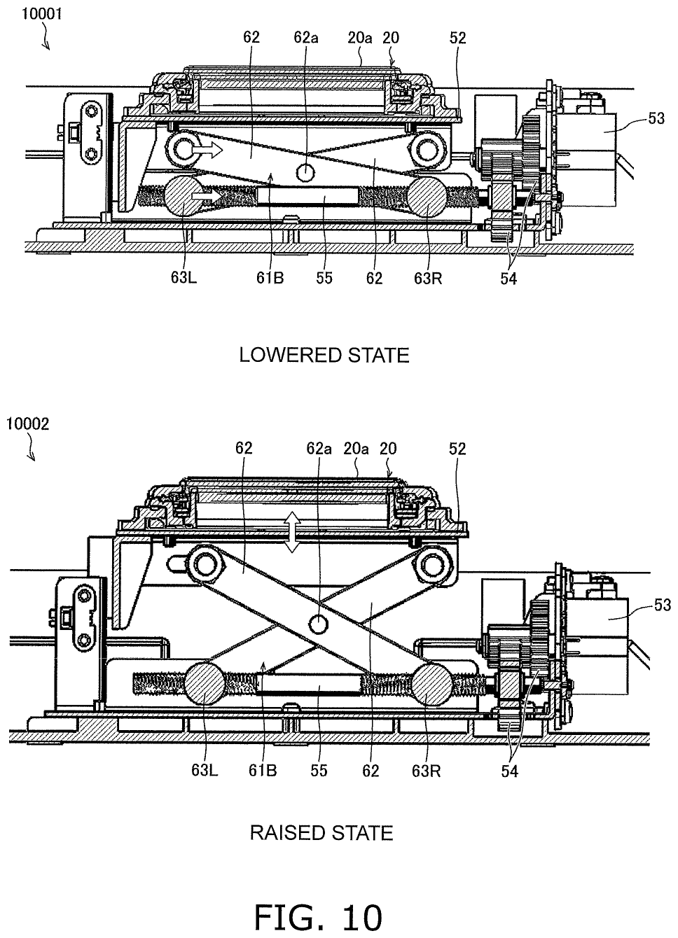

FIG. 10 is a cross-sectional view of the main components, showing the lowered state and the raised state of the pantograph mechanism 60 provided to the elevating mechanism 51 in the push button unit according to an embodiment. 10001 in the drawing shows the lowered state, and 10002 shows the raised state. As shown by 10001, in the lowered state the left round support column 63L is located at the left end, and the two frame pieces 62 of the front and back frame portions 61F and 61B extend along the ball screw 55. When the ball screw 55 rotates in a specific direction from this state, the left round support column 63L moves horizontally to the right and approaches the right round support column 63R as shown by 10002. When the left round support column 63L approaches the right round support column 63R, the two frame pieces 62 in the front and back frame portions 61F and 61B rise up in the direction away from the ball screw, and the base 52 is pushed up, resulting in the raised state.

When the ball screw 55 rotates from the raised state in the opposite direction from that when the ball screw 55 is raised, the left round support column 63L moves horizontally to the left and away from the right round support column 63R. When the left round support column 63L moves away from the right round support column 63R, the two frame pieces 62 in the front and back frame portions 61F and 61B fold toward the ball screw 55, and the base 52 is lowered, resulting in the lowered state.

In this embodiment, the amount by which the push button 20 is raised by the pantograph mechanism 60 is about 20 mm, and the reduction ratio of the reduction gears 54 is 0.1389. In this case, the elevating motor 53 has to rotate about 15 times to raise the push button 20 by 20 mm. Thus increasing the reduction ratio makes the push button 20 more resistant to sinking even if hit forcefully.

Returning to FIGS. 5 and 6, the third and fourth photosensors PS3 and PS4 sense the position of the push button 20. In this embodiment, the third and fourth photosensors PS3 and PS4 are installed on the left side, which is the opposite side from the elevating motor 53. A detection piece 56 that blocks light from reaching the third and fourth photosensors PS3 and PS4 is attached to the base 52.

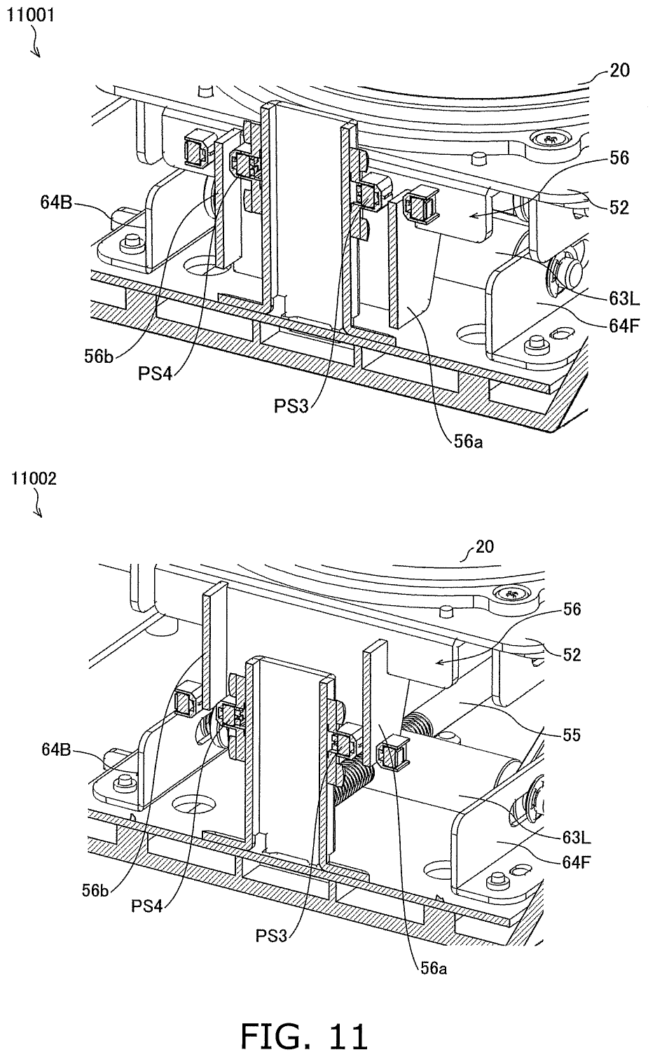

FIG. 11 is an explanatory diagram showing the sensing of the position of the push button 20 by the third and fourth photosensors PS3 and PS4 in the push button unit according to an embodiment. 11001 in the drawing shows the lowered state (in the lowered position), and 11002 shows the raised state (in the raised position). As shown in FIG. 11, the third and fourth photosensors PS3 and PS4 are installed at the same height, and the detection piece 56 has light blocking walls 56a and 56b that are offset in the up and down direction (height direction).

As by 11001 in the drawing, in the lowered state of the push button 20, the fourth photosensor PS4 is shielded from light by the light blocking wall 56b, and the third photosensor PS3 is not shielded from light by the light blocking wall 56a. When the push button 20 starts to rise, the third photosensor PS3 is also shielded from light by the light blocking wall 56a, and the third and fourth photosensors PS3 and PS4 are both in a light-shielded state.

Then, when the push button 20 reaches its raised position, as shown by 11002, the third photosensor PS3 remains shielded from light by the light blocking wall 56a, but the fourth photosensor PS4 is not shielded from light by the light blocking wall 56b. In this way, the position of the push button 20 (lowered position, raised position, or intermediate position) can be sensed from the combination in which the third and fourth photosensors PS3 and PS4 are or are not shielded from light.

### 3-3. Sensor Unit 80

The sensor unit (instruction receiving unit) 80 receives, from the outside of the push button unit 10, an instruction to execute the disinfection of the operation surface 20a with the ultraviolet irradiation unit 40. As shown in FIGS. 3 and 4, the sensor unit 80 comprises a photosensor 81 that is a reflection sensor, and is attached to the back side of the front case 12. As shown in FIG. 2, a window portion 12a through which the light of the photosensor 81 of the sensor unit 80 passes is provided at the site in the front case 12 where the sensor unit 80 is installed. The player can instruct the execution of the disinfection of the operation surface 20a in a non-contact manner by holding his or her hand over the window portion 12a.

### 4. Control Device 70

FIG. 12 is a block diagram showing a control system of the push button unit 10 according to an embodiment. The control device 70 is composed of, for example, a computer device made up of a CPU (central processing unit), an arithmetic processing unit such as a dedicated processor, and the like, and controls the operation of the various components of the push button unit 10. The control device 70 is connected to the opening and closing motor 33, the LED drive circuit 42, and the elevating motor 53 of the push button unit 10. Also, detection signals are inputted to the control device 70 from the pressure-sensitive sensor 29, the first to fourth photosensors PS1 to S4, and the sensor unit 80.

The control device 70 comprises a pressing detection unit 74, a shutter control unit 71, an irradiation control unit 72, and an elevating control unit 73. The pressing detection unit 74 determines that the push button 20 has been pressed when a detection signal is inputted from the pressure-sensitive sensor 29. When a detection signal is inputted from the sensor unit 80 (when an instruction to execute disinfection work is received), the shutter control unit 71, the irradiation control unit 72, and the elevating control unit 73 control the drive of the opening and closing motor 33, the LED drive circuit 42, and the motor 53 to execute the disinfection work.

More precisely, the shutter control unit 71 uses the detection signals of the first and second photosensors PS1 and PS2 to control the drive of the opening and closing motor 33 and to open and close the shutter 31. The irradiation control unit 72 controls the drive of the LED drive circuit 42 to turn on the disinfecting LEDs 41. The elevating control unit 73 uses the detection signals of the third and fourth photosensors PS3 and PS4 to control the drive of the elevating motor 53 and to raise and lower the push button 20.

The control device 70 may be provided with the push button unit 10, or may be provided as a part of the control device of the game machine 1 (see FIG. 1) on which the push button unit 10 is mounted.

### 5. Disinfection Operation of Push Button Unit 10

### 5-1. Main Sequence of Disinfection Operation

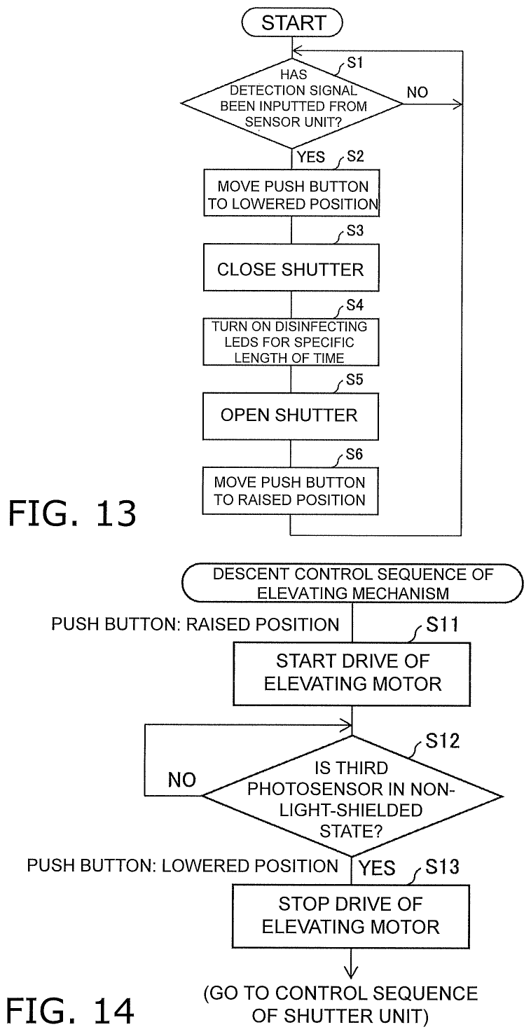

FIG. 13 is a flowchart of the main sequence of the disinfection operation in the push button unit 10 according to an embodiment. As shown in FIG. 13, the control device 70 constantly determines whether or not a detection signal has been inputted from the sensor unit 80 (S1). If it is determined in S1 that a detection signal has been inputted, the elevating control unit 73 drives the elevating motor 53 to move the push button 20 to its lowered position (S2). When the push button 20 moves to its lowered position, the shutter control unit 71 drives the opening and closing motor 33 to close the shutter 31 (S3).

When the shutter 31 closes, the irradiation control unit 72 drives the LED drive circuit 42 to turn on the disinfecting LEDs for a specific length of time (S4). This length of time is long enough for any virus adhering to the operation surface 20a to be inactivated by the ultraviolet rays from the disinfecting LEDs 41, and is set to about 10 seconds, for example. In S4, once the LEDs have been on for the specific length of time, the shutter control unit 71 drives the opening and closing motor 33 to open up the shutter 31 (S5). When the shutter 31 is opened in S5, the elevating control unit 73 drives the elevating motor 53 to move the push button 20 to its raised position (S6). After this, the control device 70 returns to the processing of S1.

The processing of S5 and S6 for opening the shutter 31 and making the push button 20 come out does not have to be performed immediately after the lighting of the disinfecting LEDs 41 for a specific length of time is completed, as long as it is after the completion of this lighting. For example, the processing of S5 and S6 may be executed at the point when it is detected that there is a situation (event) in which the push button 20 needs to be operated, such as the start of a game on the game machine or the like in which the push button unit 10 is installed.

### 5-2. Elevating Mechanism Control Sequence

FIG. 14 is a flowchart showing the descent control sequence of the elevating mechanism 51 in the push button unit 10 according to an embodiment. The descent control sequence of FIG. 14 is executed in S2 of FIG. 13.

As shown by 11002 in FIG. 11, in the raised state in which the push button 20 is in its raised position, the third photosensor PS3 is in a light-shielded state and the fourth photosensor PS4 is in a non-light-shielded state. When the elevating control unit 73 starts driving the elevating motor 53 (S11) in order to lower the push button 20 from its raised position, both the third and fourth photosensors PS3 and PS4 are in a light-shielded state.

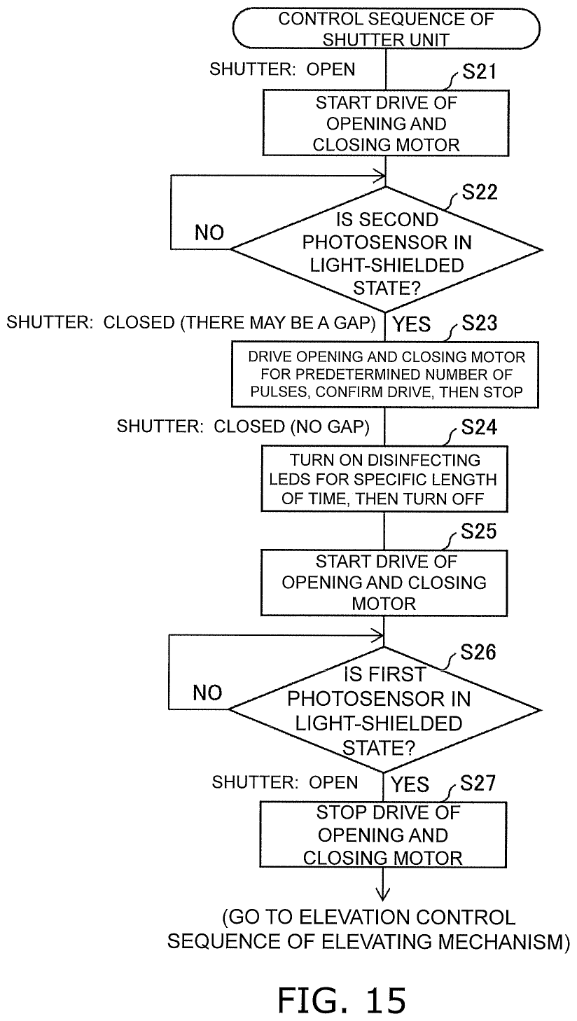

The elevating control unit 73 drives the elevating motor 53 until the third photosensor PS3 is in a non-light-shielded state. As shown by 11001 in FIG. 11, when the push button 20 reaches its lowered position, the third photosensor PS3 is in a non-light-shielded state and the fourth photosensor PS4 is in a light-shielded state. When the third photosensor PS3 is in a non-light-shielded state (Yes in S12), the elevating control unit 73 stops driving the elevating motor 53 (S13). After this, the control device 70 proceeds to the control sequence of the shutter unit 30 shown in FIG. 15.

Although not shown, the ascending control sequence for moving the push button 20 to its raised position, which is executed in S6 of FIG. 13, is the reverse of FIG. 14. That is, the elevating control unit 73 drives the elevating motor 53 to raise the push button 20 until the fourth photosensor PS4 is in a non-light-shielded state, and when the fourth photosensor PS4 is in a non-light-shielded state, the drive of the elevating motor 53 is stopped.

### 5-3. Control Sequence of Shutter Unit 30

FIG. 15 is a flowchart showing the control sequence of the shutter unit 30 in the push button unit 10 installed in the game machine 1 according to an embodiment. The control sequence in FIG. 15 is executed in S3 to S5 of FIG. 13.

As shown in FIG. 3, when the shutter 31 is in its open state (fully open), the first photosensor PS1 is in a light-shielded state and detects this fully open state. The second photosensor PS2 that detects a closed state is in a non-light-shielded state. In order to close the shutter 31, the shutter control unit 71 starts driving the opening and closing motor 33 (S21). When the shutter 31 starts to close, the first and second photosensors PS1 and PS2 are both in a non-light-shielded state. The shutter control unit 71 drives the opening and closing motor 33 until the second photosensor PS2 that detects a closed state is in a light-shielded state. As shown in FIG. 4, when the shutter 31 is closed, the second photosensor PS2 is in a light-shielded state. When the second photosensor is in a light-shielded state (Yes in S22), the shutter control unit 71 drives the opening and closing motor 33 by a specific number of pulses, confirms the drive for this specific number of pulses, and then stops (S23).

This processing of S23 is executed in order to prevent the shutter 31 from not completely closing, which would result in a tiny gap. That is, in the control to stop the opening and closing motor 33 immediately after the second photosensor PS2 is shielded from light by the right slider 37R (Yes in S22), variance in the assembly position of the second photosensor PS2 may produce a tiny gap in the closed state of the shutter 31. If S23 is executed, any variance in the assembly position of the second photosensor PS2 can be absorbed and the shutter 31 can be closed without leaving a tiny gap.

The fact that the shutter 31 is not completely closed can be detected in the following two ways. One is when the second photosensor PS2 is not shielded from light even after a specific period of time has elapsed since the opening and closing motor 33 started to be driven. The other is when it is detected that the opening and closing motor 33 has not rotated by a specific angle even after a specific period of time has elapsed even though the specified number of pulses was sent to the opening and closing motor 33 after the second photosensor PS2 is in a light-shielded state. A Hall element is provided to the motors constituting the opening and closing motor 33 and the like, and this allows the motor to confirm its own rotational position.

When the shutter 31 is completely closed by the processing of S23, the irradiation control unit 72 turns on the disinfecting LEDs 41 for a predetermined length of time and then turns them off (S24). Consequently, the operation surface 20a of the push button 20 is irradiated with ultraviolet rays, and any adhering virus is inactivated.

After confirming that the disinfecting LEDs 41 have been turned off, the shutter control unit 71 starts driving the opening and closing motor 33 in order to open the shutter 31 (S26). When the shutter 31 starts opening, the first and second photosensors PS1 and PS2 both go back into its non-light-shielded state. The shutter control unit 71 drives the opening and closing motor 33 until the first photosensor PS1 that detects an open state is back in its light-shielded state. When the first photosensor PS1 is in its light-shielded state (Yes in S26), the shutter control unit 71 stops driving the opening and closing motor 33 (S27).

### 6. Effect

With the above configuration, the push button unit 10 comprises the ultraviolet irradiation unit 40, and in a state in which the push button 20 has been retracted, the operation surface 20a is irradiated with ultraviolet rays to inactivate any virus adhering to the operation surface 20a. This makes it possible to disinfect the operation surface 20a without relying on disinfection work in which a worker applies a disinfectant solution, freeing the worker from frequent disinfection work and reducing the burden on the worker. Also, since no disinfectant solution is used, the operation surface 20a will not be deteriorated by disinfectant solution, and the service life of the push button 20 can be extended.

Also, because a player can recognize that the operation surface 20a has been disinfected in its retracted state, the player can confirm that the push button 20 has been disinfected just by seeing the retracted state. Consequently, the notion of the button having been disinfected is more appealing to the player than a push button made of a material containing an antibacterial agent, and the player can rest assured that any virus has been inactivated.

Also, with the above configuration, the ultraviolet irradiation by the ultraviolet irradiation unit 40 is performed in a state in which the shutter 31 has been completely closed. This reliably prevents the player from looking at the emitted ultraviolet rays.

Also, with the above configuration, the elevating mechanism 51 for raising and lowering the push button 20 is provided, and the shutter 31 closes off the opening 14 in a state in which the push button 20 is lowered. With this configuration, the shutter 31 can have a flat configuration, and the push button unit 10 can be thinner. This allows for installation in a limited space such as the operation unit 3 of the game machine 1 (see FIG. 1).

Also, with the above configuration, the elevating mechanism 51 comprises a pantograph mechanism that raises and lowers the base 52 using the drive force of the elevating motor 53. Using the pantograph mechanism increases the reduction ratio of the reduction gear 54 that transmits the drive force of the elevating motor 53 to the pantograph mechanism, so the push button 20 can be held in its raised position even if a large force is applied to the push button 20.

Also, with the above configuration, since the sensor unit 80 is designed to receive an instruction to execute the disinfection operation at any point in time, the player can disinfect the push button whenever desired. In particular, with the above configuration, since the sensor unit 80 comprises a reflection sensor, a player can issue a command to execute a disinfection operation on the push button in a non-contact manner.

### (3) Modification Examples

An embodiment of the present invention was described in detail above, but the above description is merely an example of the present invention in all respects. It should go without saying that various improvements and modifications are possible without departing from the scope of the present invention. For instance, the following modifications can be made. In the following, Constituent elements that are the same as in the above embodiment will be numbered the same, and points that are the same as in the above embodiment may be omitted as appropriate. The following modification examples can be combined as appropriate.

3.1

In the main sequence of the disinfection operation shown in FIG. 13, the control device 70 constantly determines whether or not a detection signal has been inputted from the sensor unit 80 in S1, and if it is determined that a detection signal has been inputted, S2 is executed to start the disinfection operation. That is, with the configuration of the embodiment described above, the instruction inputted by the player to the sensor unit 80 was an event that triggered the execution of a predetermined disinfection operation.

However, the instruction to execute the disinfection operation by the sensor unit 80 is only an example of the above-mentioned trigger. For instance, the completion of the operation on the push button 20 may be detected by some method, and this may serve as a trigger. For example, in the case of installation in the game machine 1, the end of a game on this game machine can be an event that triggers the execution of the disinfection operation. However, if the push button unit 10 is installed in the game machine 1, the event that triggers the execution of the disinfection operation is preferably a player change.

3.2

When the push button unit 10 is installed in the game machine 1 (see FIG. 1), the installation space is limited, so the push button unit 10 needs to have a thin configuration. Therefore, the elevating mechanism 51 is provided to lower the push button 20, and the push button 20 is covered by the shutter 31 in its lowered state. With this configuration, the shutter can be configured in a flat shape such as that of the shutter 31, and the thickness of the push button unit 10 can be reduced. On the other hand, if there is plenty of installation space, the elevating mechanism 51 need not be provided, and the shutter may be three-dimensionally configured to cover the push button 20.

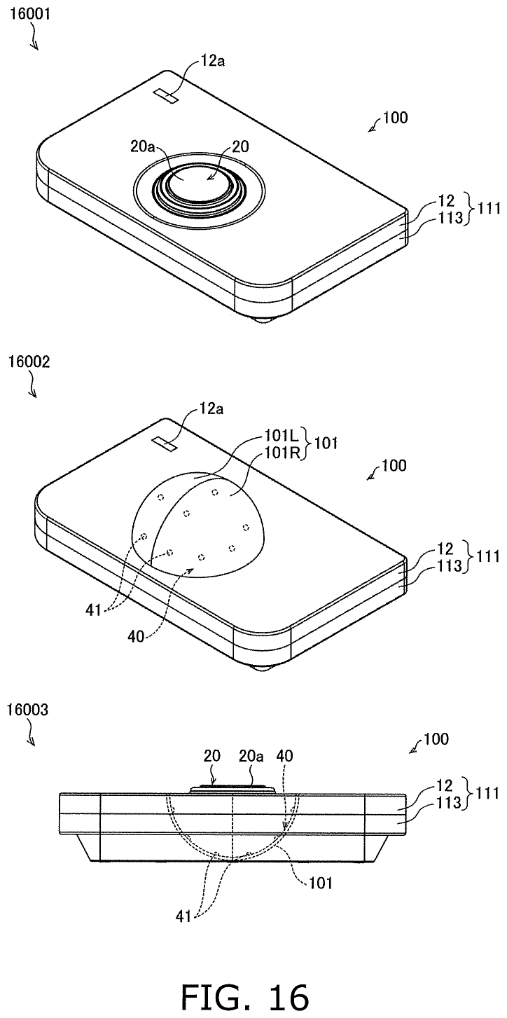

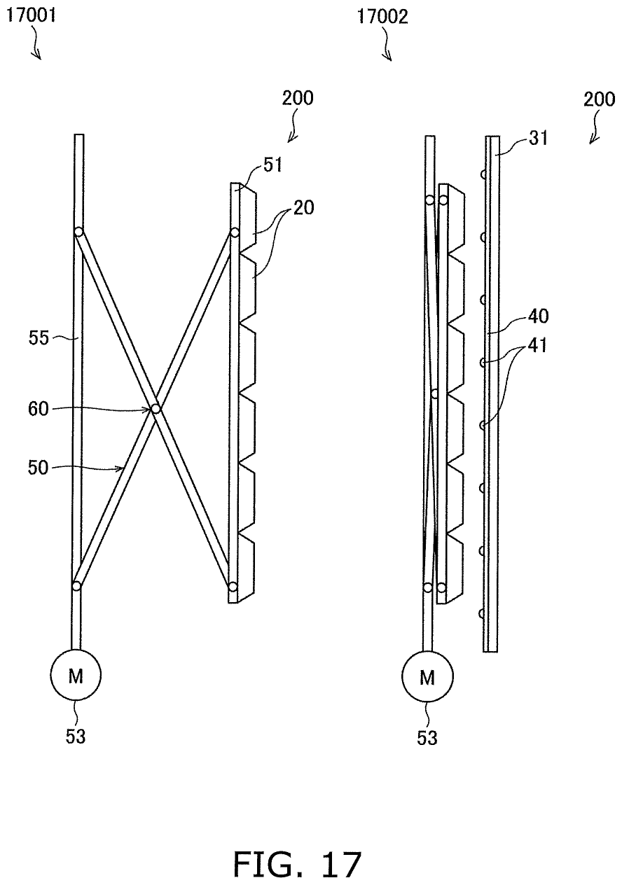

FIG. 16 is a diagram illustrating the push button unit 100 according to a modification example, and shows the normal state in which the push button 20 has come out, and the retracted state in which the push button 20 has been retracted. 16001 and 16003 show the normal state in the drawing, and 16002 shows the retracted state.

As shown in FIG. 16, the push button unit 100 comprises a dome-shaped three-dimensional shutter 101 instead of the flat shutter 31 used in the push button unit 10. The shutter 101 is made up of left and right shutters 101L and 101R. An ultraviolet irradiation unit (light irradiation unit) 40 is installed on the back surface of the shutter 101. Using the three-dimensional shutter 101 eliminates the need to raise and lower the push button 20, so there is no need to provide the elevating mechanism 51.

3.3

A configuration comprising one push button 20 was given above as an example of the push button unit 10, but the configuration may also comprise a plurality of push buttons 20. The push button unit 10 comprising a plurality of push buttons 20 is installed in an elevator or a ticket vending machine, for example.

FIG. 17 is a cross-sectional view of the main components of a push button unit 200 comprising a plurality of push buttons 20 according to a modification example, and shows the normal state in which the push buttons have come out and the retracted state in which the push buttons have been retracted. 17001 shows the normal state, and 17002 shows the retracted state.

In the push button unit 200, the plurality of push buttons 20 are mounted on a base 52, and the base 52 is raised and lowered by the elevating mechanism 51. The shutter 31 opens and closes to the left and right, or up and down, and the ultraviolet irradiation unit 40 is installed on the back surface of the shutter 31. Here again, the sensor unit 80 may be mounted and the disinfection operation may be executed at the desired timing.

Also, in the case of an elevator, for example, the disinfection operation may be executed when it is detected from the detection result of a weight sensor or the like that nobody is aboard. Also, in the case of a ticket vending machine comprising a touch panel, the disinfection operation may be executed after a ticket has been issued. The detection that nobody is aboard, and the completion of a ticket sale correspond to events that trigger a disinfection operation.

Also, a shutter unit comprising a three-dimensional shutter 101 provided with an ultraviolet irradiation unit 40 on the back surface and a shutter mechanism 32 for opening and closing the shutter 101 may be provided so as to be externally attached to an existing push button 20.

The present invention is not limited to the embodiments given above, and various modifications are possible within the scope of the claims, and any embodiments obtained by suitably combining the technical means disclosed in different embodiments are also encompassed by the technical scope of the present invention.

1. A push button unit, comprising:
a push button;
a shutter configured to cover the push button in a closed state;
a shutter mechanism configured to open and close the shutter; and
a light irradiation unit having a light source that is disposed on a back surface of the shutter and is opposite an operation surface of the push button in the closed state of the shutter, and emits a light beam configured to inactivate a virus.

2. The push button unit according to claim 1, further comprising:
a panel having an opening through which the push button comes out; and
an elevating mechanism configured to raise and lower the push button,
wherein the push button comes out through the opening at a raised position, and
the shutter is disposed on a back surface of the panel and blocks off the push button into a closed state, with the push button in a lowered position.

3. The push button unit according to claim 2,
wherein the elevating mechanism comprises:
a motor; and
a pantograph mechanism configured to move a base on which the push button is mounted between a lowered position and a raised position by means of a drive force of the motor.

4. The push button unit according to claim 1, further comprising an instruction receiving unit configured to receive, at an arbitrary timing, an instruction to execute a disinfection operation that inactivates any virus adhering to the operation surface with the light beam.

5. The push button unit according to claim 4, wherein the instruction receiving unit comprises a reflection sensor.

6. A control device for the push button unit according to claim 1,
wherein, when an event occurs that triggers a disinfection operation that inactivates any virus adhering to the operation surface occurs with a light beam, the control device controls a drive of the shutter mechanism to put the shutter in the closed state, controls a drive of the light irradiation unit to turn on the light source for a specific length of time, and after this lighting is completed, controls the drive of the shutter mechanism to put the shutter in an open state.

7. A control device for the push button unit according to claim 2,
wherein, when an event occurs that triggers a disinfection operation that inactivates any virus adhering to the operation surface occurs with a light beam, the control device controls a drive of the elevating mechanism to lower the push button, and controls a drive of the shutter mechanism to put the shutter in the closed state, and then controls a drive of the light irradiation unit to turn on the light source for a specific length of time, and after this lighting is completed, controls the drive of the shutter mechanism to put the shutter in an open state, and controls the drive of the elevating mechanism to raise the push button.
